(19) United States
(12) Patent Application Publication
(10) Pub. No.: US 2023/0338219 A1
(43) Pub. Date: Oct. 26, 2023

(54) INTELLIGENT MOUTH OPENING REHABILITATION DEVICE

(71) Applicant: Shanghai Ninth People's Hospital, Shanghai JiaoTong University School of Medicine, Shanghai (CN)

(72) Inventors: Jiannan LIU, Shanghai (CN); Guangtao ZHAI, Shanghai (CN); Chenping ZHANG, Shanghai (CN); Zhiyuan ZHANG, Shanghai (CN)

(73) Assignee: Shanghai Ninth People's Hospital, Shanghai JiaoTong University School of Medicine, Shanghai (CN)

(21) Appl. No.: 18/027,637

(22) PCT Filed: Jul. 27, 2021
(86) PCT No.: PCT/CN2021/108569

§ 371 Date: Mar. 22, 2023

(30) Foreign Application Priority Data
Sep. 22, 2020 (CN) 2020110023113

(51) Int. Cl. A61H 1/02

(52) U.S. Cl. CPC A61H 1/02; A61H 2201/5071; A61H 2201/5069; A61H 2205/022; A61H 2201/5046; A61H 2201/501

(57) ABSTRACT

An intelligent mouth opening rehabilitation device is provided, including: a body, an end of the body is provided with an upper bite pad and a lower bite pad; a pressure sensor, for detecting pressure data on the upper bite pad and/or the lower bite pad; a stepping motor, for driving the upper bite pad and/or the lower bite pad to swing longitudinally; and a processor, for controlling the stepping motor to operate or stop according to the pressure data and/or a preset training instruction, thereby driving the upper bite pad and/or the lower bite pad to swing longitudinally or to stop. Compared with traditional mouth opening rehabilitation devices, the intelligent mouth opening rehabilitation device controls automatically the upper bite pad and/or the lower bite pad to swing longitudinally, so that a mouth opening training is more accurate, and more controllable. Therefore, a rehabilitation effect is greatly improved.

### CROSS REFERENCE TO RELATED APPLICATION

This is a Sect. 371 National Stage application of a PCT International Application No. PCT/CN2021/108569, filed on Jul. 27, 2021, which claims priority to Chinese Patent Application No. 2020110023113, filed with the China National Intellectual Property Administration on Sep. 22, 2020 and entitled “INTELLIGENT MOUTH OPENING REHABILITATION DEVICE”, which is incorporated herein by reference in its entirety.

### TECHNICAL FIELD

The present disclosure relates to the field of oral rehabilitation medical instruments, and in particular, to an intelligent mouth opening rehabilitation device.

### BACKGROUND

A limited mouth opening is one of the most common complications in patients with head and neck cancer after comprehensive treatment. This not only affects a quality of life of patients and even threatens their lives, but also brings a serious social and economic burden. So it is very urgent for oral and maxillofacial surgeons to solve a problem that patients have difficulty in opening their mouth after being treated.

Currently, the main principles of treating the limited mouth opening are to control the progress of the limited mouth opening and to restore functions. However, several approaches to the treatment of mouth opening difficulties have been evaluated by scholars, and current clinical evidences suggest that a maximum mouth opening angle of patients with a mouth opening training intervention or using a mouth opening rehabilitation device is better than that of patients without assisted exercise.

At present, some mouth opening rehabilitation devices are existed on the market. For example, a DynBite temporomandibular joint dynamic stretching rehabilitation device is a rehabilitation product developed by Suzhou MicroPort Ric MedTech Ltd. This rehabilitation device is used to treat patients with the limited mandibular opening caused by muscle injury, surgery, tumor radiation therapy, temporomandibular joint dysfunction, osteoarthritis, post-infection or trauma. The rehabilitation device can relieve patients' difficulty in mouth opening and help them obtain a larger mouth opening angle, thereby restoring normal functions of the temporomandibular joint.

However, most mouth opening rehabilitation devices are still in traditional manual operation modes such as manual measurement, manual recording, etc. So it is very urgent to provide an intelligent mouth-opening rehabilitation device that automatically opens a training angle, automatically obtains a degree of force, automatically assesses pain, automatically records training data and so on.

### SUMMARY

The present disclosure provides an intelligent mouth opening rehabilitation device.

The intelligent mouth opening rehabilitation device includes: a body, an end of the body being provided with an upper bite pad and a lower bite pad; a pressure sensor, provided in the upper bite pad and/or the lower bite pad, for detecting pressure data on the upper bite pad and/or the lower bite pad; a stepping motor, for driving the upper bite pad and/or the lower bite pad to swing longitudinally; and a processor, for controlling the stepping motor to operate or stop according to the pressure data and/or a preset training instruction, thereby driving the upper bite pad and/or the lower bite pad to swing longitudinally or to stop.

In an embodiment, the processor controls the stepping motor to operate or stop according to the pressure data, including: the processor compares the pressure data with a preset pressure threshold in real time; if the pressure data are lower than the preset pressure threshold, the processor controls the stepping motor to operate forward, to enable the upper bite pad to swing up a distance and/or the lower bite pad to swing down a distance, then returns to the first operation; and if the pressure data are not lower than the preset pressure threshold, the processor controls the stepping motor to stop operating, to enable the upper bite pad and/or the lower bite pad to stop swinging.

In an embodiment, the processor controls the stepping motor to operate or stop according to the pressure data, including: detecting the pressure data in real time to determine whether the pressure data reach a second pressure threshold, where the second pressure threshold is greater than the preset pressure threshold; and if the pressure data reach the second pressure threshold, the processor controls the stepping motor to operate reversely, thereby enabling the upper bite pad to swing down a distance and/or the lower bite pad to swing up a distance, then returns to the first operation.

In an embodiment, the preset training instruction includes: a preset training duration, and a preset opening angle/a preset opening distance between the upper bite pad and the lower bite pad.

In an embodiment, an angle sensor is provided in the upper bite pad and/or the lower bite pad and used to collect and characterize an opening angle between the upper bite pad and the lower bite pad; or an infrared rangefinder is provided in the upper bite pad and/or the lower bite pad and used to collect and characterize an opening distance between the upper bite pad and the lower bite pad.

In an embodiment, the processor controls the stepping motor to operate or stop according to the preset opening angle or the preset opening distance in the preset training instruction, including: the processor controls the stepping motor to operate forward according to the received preset opening angle or preset opening distance, to enable the upper bite pad to swing up a distance and/or the lower bite pad to swing down a distance; and when the angle sensor detects that the opening angle between the upper bite pad and the lower bite pad reaches the preset opening angle, or when the infrared rangefinder detects that the opening distance between the upper bite pad and the lower bite pad reaches the preset opening distance, the processor controls the stepping motor to stop operating.

In an embodiment, the processor controls the stepping motor to operate or stop according to the preset training duration in the preset training instruction, including: when the stepping motor starts and enters a stop state, a countdown is carried out based on the preset training duration; and when the timer detects that the training duration has been reached, a speaker emits a prompt tone, and the processor controls the stepping motor to operate reversely, so that the upper bite pad swings down to its starting position and/or the lower bite pad swings up to its starting position.

In an embodiment, the intelligent mouth opening rehabilitation device further includes: a light emitting diode (LED) touch screen, located on one side of the body, used to display real-time pressure data, a training duration, training times, and an opening angle/an opening distance, and/or, used to manually set preset training times and the preset opening angle or the preset opening distance; and a communicator, communicating with an external device, used to send the real-time pressure data, the training duration, the training times, and the opening angle/the opening distance to the external device, and/or, used to receive the preset training times and the preset opening angle or the preset opening distance from the external device.

In an embodiment, a communication mode of the communicator includes a wired communication mode and/or a wireless communication mode; the wired communication mode includes one or a combination of USB1.0/2.0/3.x, Micro USB, Mini USB, serial interface and parallel interface; the wireless communication mode includes one or a combination of 2G/3G/4G/5G, Bluetooth, Infrared, NB-IoT, Rola, Zigbee, MavLink, WIFI, NFC, GPRS, GSM and Ethernet.

In an embodiment, the upper bite pad and the lower bite pad are both provided with a corresponding stepping motor respectively; or, one of the upper bite pad and the lower bite pad is provided with a corresponding stepping motor and the other of the upper bite pad and the lower bite pad is fixed.

As described above, the present disclosure provides an intelligent mouth opening rehabilitation device. The intelligent mouth opening rehabilitation device includes: a body, an end of the body being provided with an upper bite pad and a lower bite pad; a pressure sensor, provided in the upper bite pad and/or the lower bite pad, for detecting pressure data on the upper bite pad and/or the lower bite pad; a stepping motor, for driving the upper bite pad and/or the lower bite pad to swing longitudinally; and a processor, for controlling the stepping motor to operate or stop according to the pressure data and/or a preset training instruction, thereby driving the upper bite pad and/or the lower bite pad to swing longitudinally or to stop.

The Following Beneficial Effects are Achieved:

Compared with traditional mouth opening rehabilitation devices that require manual measurement of the opening angle or opening distance per use and require the need to manually control the training duration, the intelligent mouth opening rehabilitation device of the present disclosure controls automatically the upper bite pad and/or the lower bite pad to swing longitudinally, so that the mouth opening training is more accurate, more scientific and more controllable. Therefore, a rehabilitation effect may be greatly improved by using the intelligent mouth opening rehabilitation device in the present disclosure.

### BRIEF DESCRIPTION OF THE DRAWINGS

FIG. 1 is a side structural diagram of an intelligent mouth opening rehabilitation device according to an embodiment of the present disclosure.

FIG. 2 is an overall structural diagram of an intelligent mouth opening rehabilitation device according to an embodiment of the present disclosure.

### DETAILED DESCRIPTION OF THE PREFERRED EMBODIMENTS

The embodiments of the present disclosure will be described below through specific examples. One skilled in the art can easily understand other advantages and effects of the present disclosure according to contents disclosed in the description. The present disclosure may also be implemented or applied through other different embodiments, and various modifications or changes may be made to all details in the description based on different points of view and applications without departing from the spirit of the present disclosure. It should be noted that the embodiments of the present disclosure and the features of the present disclosure can be combined with each other without conflict.

The embodiments of the present disclosure will be described in detail below with reference to the accompanying drawings, so that those skilled in the art to which the present disclosure pertains can easily implement. The present disclosure can be embodied in many different forms, and is not limited to the embodiments described herein.

In order to clearly describe the present application, components irrelevant to the description are omitted, and the same or similar components throughout the specification are given the same reference numerals.

Throughout the specification, when a component is said to be “connected” to another component, this includes not only the case of “direct connection”, but also the case of “indirect connection” with other elements interposed there between. In addition, when a component is said to “include” a certain constituent element, unless there is particularly no description to the contrary, it does not exclude other constituent elements, but means that other constituent elements may also be included.

When an element is said to be “on” another element, it can be directly on the other element, but it can also be accompanied by other elements in between. When an element is referred to as being “directly on” another element, it is not accompanied by the other element in between.

Indications for the intelligent mouth opening rehabilitation device described in the present disclosure include but are not limited to: temporomandibular arthritis, arthroscopy, joint fibrosis, disuse atrophy, joint replacement, facial muscle disease, articular disc excision, radiation therapy, reconstructive surgery, temporomandibular joint disorders, trauma, trismus. Contraindications for the intelligent mouth opening rehabilitation device include fractures of the mandible or maxilla, infection, osteomyelitis, and osteonecrosis of the jaw. After the intelligent mouth opening rehabilitation device described in the present disclosure is used by patients, the range of motion of the mandible is increased, functions of the mandible and joints are improved, the limited mouth opening caused by cancer, trauma, temporomandibular joint disease, facial burns and stroke is significantly relieved, swelling and pain are also reduced.

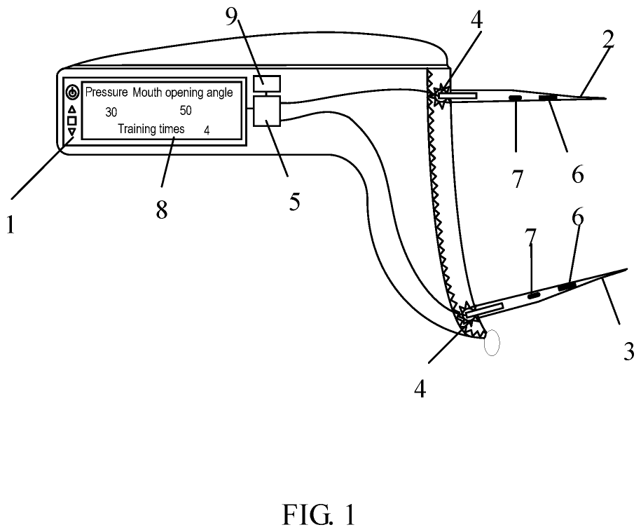

FIG. 1 is a side structural diagram of an intelligent mouth opening rehabilitation device according to an embodiment of the present disclosure. As shown in FIG. 1, the intelligent mouth opening rehabilitation device includes a body 1. An end of the body 1 is provided with an upper bite pad 2 and a lower bite pad 3.

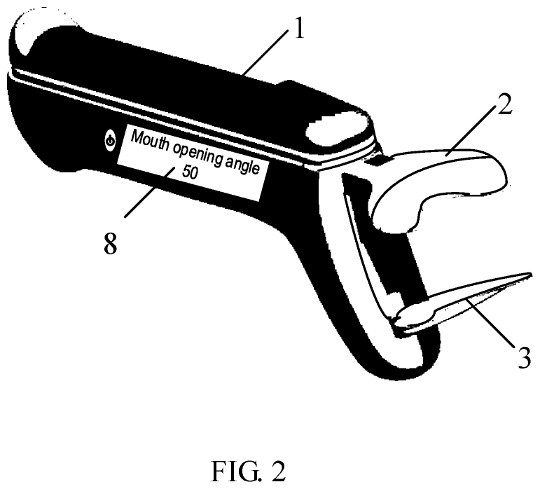

In one or more embodiments, the body 1 is L-shaped or pistol-shaped, and the body 1 includes a transverse part and a longitudinal part. Where the transverse part of the body 1 includes a hand holding part and a light-emitting diode (LED) display part. The longitudinal part of the body 1 corresponds to a bite assembly placed in a user's mouth for a mouth opening training, and is also a teeth supporting part that enters the interior of the user's mouth. In the present disclosure, the above structure of the body 1 fully considers the design principle of ergonomics, the hand holding part is round and full as a whole, the partially sunken human-machine curved surface guides a grasping position of a palm, and a rear end of the hand holding part is designed with an anti-slip surface. FIG. 2 is an overall structural diagram of an intelligent mouth opening rehabilitation device according to an embodiment of the present disclosure.

In an embodiment, the body 1 includes a pressure sensor 6 and a stepping motor 4. The pressure sensor 6 is provided in the upper bite pad 2 and/or the lower bite pad 3, and is for detecting pressure data on the upper bite pad 2 and/or the lower bite pad 3. The stepping motor 4 is for driving the upper bite pad 2 and/or the lower bite pad 3 to swing longitudinally.

In one or more embodiments, a manner in which the stepping motor 4 drives the upper bite pad 2 and/or the lower bite pad 3 to achieve a longitudinal swing includes various common manners. In an embodiment, as shown in FIG. 1, the stepping motor 4 drives a gear to rotate, thereby driving the upper bite pad 2 and/or the lower bite pad 3 to move. In another embodiment, the stepping motor 4 drives a lever, thereby driving the upper bite pad 2 and/or the lower bite pad 3 to swing. Manners of driving the upper bite pad 2 and/or the lower bite pad 3 are not limited in the present disclosure.

In an embodiment, the pressure sensor 6 is only provided in the upper bite pad 2, the stepping motor 4 is set correspondingly for the upper bite pad 2, the lower bite pad 3 is fixed (that is, the lower bite pad 3 may not move or swing), and the stepping motor 4 corresponding to the lower bite pad 3 is not provided. The above structure enables the processor 5 to only control the upper bite pad 2 to swing longitudinally or to stop. In another embodiment, the pressure sensor 6 is only provided in the lower bite pad 3, the stepping motor 4 is set correspondingly for the lower bite pad 3, the upper bite pad 2 is fixed (that is, the upper bite pad 2 may not move or swing), and the stepping motor 4 corresponding to the upper bite pad 2 is not provided. The above structure enables the processor 5 to only control the lower bite pad 3 to swing longitudinally or to stop. In other embodiments, both the upper bite pad 2 and the lower bite pad 3 are provided with the pressure sensors 6 respectively, and the upper bite pad 2 and the lower bite pad 3 are provided with the stepping motors 4 respectively. The above structure enables the processor 5 to simultaneously control the upper bite pad 2 and the lower bite pad 3 to swing longitudinally or to stop, or control the upper bite pad 2 or the lower bite pad 3 to swing longitudinally or to stop.

In addition, existing mouth opening rehabilitation devices do not have a pressure detection device, so during the mouth opening training, patients may only perceive the presence of pain or an opening angle by themselves. However, the pain degree described by patients is not accurate enough for doctors to judge the rehabilitation effect or to formulate the mouth opening training. In addition, most mouth-opening rehabilitation devices have simple mechanical structures without space or foundation to add the pressure sensor 6. In the present disclosure, the pressure sensor 6 detects pressure data on the upper bite pad 2 and/or the lower bite pad 3, and pressure data are displayed on a LED touch screen 8. During the mouth opening training, the doctor may assess the pain degree of the patient at any time according to pressure data displayed on the LED touch screen 8, and then adjust the opening degree of the training and evaluate the degree of rehabilitation. Therefore, an effect of the mouth opening training may be easily measured by the doctors.

The body 1 includes the processor 5. The processor 5 controls the stepping motor 4 to move or stop according to the pressure data and/or a preset training instruction, thereby driving the upper bite pad 2 or the lower bite pad 3 to swing longitudinally or to stop.

In an embodiment, the processor 5 controls the stepping motor 4 to move or stop according to the pressure data, including the following operations:

Operation A, the processor compares the pressure data with a preset pressure threshold in real time.

Optionally, the preset pressure threshold may be a preset pressure threshold for a specific patient obtained by testing the patient's pain performance in combination with the physician's observations when the physician instructs the patient on the use of the mouth opening rehabilitation device in an early stage.

Operation B, if the pressure data are lower than the preset pressure threshold, the processor 5 controls the stepping motor 4 to operate forward, to enable the upper bite pad 2 to swing up a distance and/or the lower bite pad 3 to swing down a distance, then, returning to Operation A.

It should be noted that the distance mentioned here is preferably a driving distance generated by one or more pulse signals of the stepping motor 4. In practice, the distance is less than 10 mm.

Operation C, if the pressure data are not lower than the preset pressure threshold, the processor 5 controls the stepping motor 4 to stop operating, to enable the upper bite pad 2 and/or the lower bite pad 3 to stop swinging. That is, the stepping motor 4 stops operating, then the upper bite pad 2 and/or the lower bite pad 3 are fixed in the current swing state.

When the upper bite pad 2 and/or the lower bite pad 3 swings to be in contact with the teeth of the patient, the pressure data detected by the pressure sensor 6 on the upper bite pad 2 and/or the lower bite pad 3 are increased gradually. So it can be determined that the upper bite pad 2 and/or the lower bite pad 3 has come into contact with the teeth of the patient according to an increasing pressure data, i.e., essentially at the patient's maximum angle of mouth opening. Therefore, the stepping motor 4 stops operating, so that the upper bite pad 2 and/or the lower bite pad 3 are fixed in the current swing state. The rehabilitation training for the patient's mouth opening is realized finally.

For example, when the intelligent mouth opening rehabilitation device is not started, the opening angle between the upper bite pad 2 and the lower bite pad 3 is zero. After the intelligent mouth opening rehabilitation device is sterilized, it may be put into the patient's mouth, then the intelligent mouth opening rehabilitation device is activated. If the training instruction is not set or received from the external device, the intelligent mouth opening rehabilitation device operates by controlling the stepping motor 4 to operate or to stop according to a default pressure data. The pressure sensor 6 provided in the upper bite pad 2 and/or the lower bite pad 3 detects the pressure data in real time. If the pressure data are lower than the preset pressure threshold, the stepping motor 4 operates forward, to enable the upper bite pad 2 to swing up a distance and/or the lower bite pad 3 to swing down a distance. That is, the opening angle between the upper bite pad 2 and the lower bite pad 3 is increased gradually. When the opening angle reaches the maximum angle that the patient's mouth may open, the resistance on the upper bite pad 2 and the lower bite pad 3 will increase continuously, so the pressure data detected by the pressure sensor 6 are also increased continuously. If the pressure data reach the preset pressure threshold, the stepping motor 4 stops operating. So far, one mouth opening training is finished based on the patient's current opening angle.

When the patient wants to interrupt the mouth opening training, or to reduce the opening angle due to increasing soreness of the oral jaw muscles. In order to make this intelligent mouth opening rehabilitation device more humanized, the processor 5 controlling the stepping motor 4 to operate or stop according to the pressure data further includes:

Operation A, the pressure sensor detects the pressure data in real time to determine whether the pressure data reach a second pressure threshold. Where the second pressure threshold is greater than the preset pressure threshold.

Operation B, if the pressure data reach the second pressure threshold, the processor 5 controls the stepping motor 4 to operate reversely, thereby enabling the upper bite pad 2 to swing down a distance and/or the lower bite pad 3 to swing up a distance, and returning to Operation A.

When the patient wants to interrupt the mouth opening training, the patient may exert a pressure on the upper bite pad 2 and the lower bite pad 3 through the upper and lower jaws of the mouth to reduce the opening angle. Or when the patient wants to reduce the opening angle due to increasing soreness of the oral-jaw muscles after a long period of fixation, the patient may exert a pressure on the upper bite pad 2 and the lower bite pad 3 through the upper and lower jaws of the mouth to reduce the opening angle.

In an embodiment, the training instruction includes: a preset training duration, and/or a preset opening angle or a preset opening distance between the upper bite pad 2 and the lower bite pad 3.

It should be noted that the training instruction may be a reasonable and scientific training setting or planning set by doctors or patients according to the difficulty of mouth opening. For example, doctors or patients may increase the opening angle gradually or increase the training duration gradually according to patients' rehabilitation. So the training instruction may be used not only for a single training, but also for a quantitative, timed, long-term training program in the rehabilitation cycles. Because the intelligent mouth opening rehabilitation device and the remote device may communicate with each other through the training data (e.g., training data include pressure peaks, training times, the opening angle/the opening distance for each or more trainings), and doctors can obtain training data received in the background and provide real-time feedback and advice on the patient's training at home with this intelligent mouth opening rehabilitation device. For example, the intelligent mouth opening rehabilitation device sends training data to an application (APP), and the APP sends feedback (e.g., diet advice, training level advice) automatically to patients according training data.

Compared with traditional mouth opening rehabilitation devices that require manual measurement of the opening angle or opening distance per use and require the need to manually control the training duration, the intelligent mouth opening rehabilitation device in the present disclosure controls automatically the upper bite pad and/or the lower bite pad to swing longitudinally, so that the mouth opening training is more accurate, more scientific and more controllable. Therefore, the rehabilitation effect can be greatly improved by using the intelligent mouth opening rehabilitation device in the present disclosure.

Referring to FIG. 1, a reference numeral 7 represents an angle sensor, or an infrared rangefinder. For ease of understanding, both the angle sensor and the infrared rangefinder described in the present disclosure are represented by the reference numeral 7.

In an embodiment, the angle sensor 7 is provided in the upper bite pad 2 and/or the lower bite pad 3, and used to detect the opening angle between the upper bite pad 2 and the lower bite pad 3. In another embodiment, the infrared rangefinder 7 is provided in the upper bite pad 2 and/or the lower bite pad 3, and used to detect the opening distance between the upper bite pad 2 and the lower bite pad 3.

The opening angle or the opening distance indicates the size of the patient's mouth opening, which indirectly reflects the difficulty of the patient's mouth opening. Based on this, the opening angle or the opening distance may be gradually increased to help patients restore their mouth opening scientifically and reasonably.

In an embodiment, the processor 5 controls the stepping motor 4 to operate or stop according to the preset opening angle or the preset opening distance in the preset training instruction, which includes:

Operation A, the processor 5 controls the stepping motor 4 to operate forward according to the preset opening angle or the preset opening distance, to enable the upper bite pad 2 to swing up a distance and/or the lower bite pad 3 to swing down a distance.

Operation B, when the angle sensor 7 detects that the opening angle between the upper bite pad 2 and the lower bite pad 3 reaches the preset opening angle, or when the infrared rangefinder 7 detects that the opening distance between the upper bite pad 2 and the lower bite pad 3 reaches the preset opening distance, the processor 5 controls the stepping motor 4 to stop operating.

In an embodiment, the processor 5 controls the stepping motor 4 to operate or stop according to the preset training duration in the preset training instruction, which includes:
Operation A, when the stepping motor starts and enters a stop state, a countdown is carried out based on the preset training duration;
Operation B, when the timer detects that the training duration has been reached, a speaker emits a prompt tone, and the processor 5 controls the stepping motor 4 to operate reversely, so that the upper bite pad 2 swings down to its starting position and/or the lower bite pad 3 swings up to its starting position.

In an embodiment, the processor 5 controls the stepping motor 4 to operate or stop according to one of the preset opening angle/the preset opening distance or the preset training duration in the preset training instruction. In another embodiment, the processor 5 also controls the stepping motor 4 to operate or stop according to a combination of the preset opening angle or preset opening distance with the preset training duration in the preset training instruction.

For example, the processor 5 first controls the stepping motor 4 to enable the upper bite pad 2 and/or the lower bite pad 3 to swing to a corresponding opening angle or a corresponding opening distance according to the preset opening angle or the preset opening distance, and then controls the stepping motor 4 to fix or pause the swing angle of the upper bite pad 2 and/or the lower bite pad 3 according to the preset training duration. Based on above processes, the mouth opening training for patients is realized.

In an embodiment, the intelligent mouth opening rehabilitation device includes a light emitting diode (LED) touch screen 8 and a communicator 9. The light emitting diode touch screen 8 is located on one side of the body 1, and used to display real-time pressure data, a training duration, training times, and an opening angle/an opening distance; and/or, used to manually set preset training times and the preset opening angle or the preset opening distance. The communicator 9 communicates with an external device and used to send the real-time pressure data, the training duration, the training times, and the opening angle/the opening distance to the external device; and/or, used to receive preset training times and the preset opening angle/the preset opening distance from the external device.

The training instruction may be manually set through the LED touch screen 8, or input from the external device through the communicator 9.

The communicator 9 communicates with the external device to transmit pressure data. In an embodiment, the external device may be smart phones, smart watches, training applications, hospital data systems, training data platforms and the like of the doctors or patients. So doctors or systems can assess the patient's training and rehabilitation progress, and provide advice, according to training data recorded remotely by the external device. Or, training data may be synchronized to a mobile device at any time. The user may read the data and modify the information of the intelligent mouth opening rehabilitation device through an applet or APP, and upgrade a system of the intelligent mouth opening rehabilitation device through Over-the-Air Technology (OTA) to optimize functions and fix bugs.

For example, if a patient has severe pain during the process of using the intelligent mouth opening rehabilitation device, the pain information may be immediately sent to the doctor through the APP and the patient is also informed to stop training. For example, The APP will record the degree of mouth opening before and after each training, provide dietary advice, and conduct regular assessments of the degree of mouth opening training. The doctor will conduct real-time monitoring through a background and provide timely guidance. The LED touch screen 8 is used for real-time self-control of the patient during the process of the mouth opening training.

In an embodiment, a communication mode of the communicator 9 includes a wired communication mode and/or a wireless communication mode. The wired communication mode includes one or a combination of USB1.0/2.0/3.x, Micro USB, Mini USB, serial interface and parallel interface (e.g., RS485, Standard Parallel Port (SPP)). The wireless communication mode includes one or a combination of 2G/3G/4G/5G, Bluetooth, Infrared, Narrow Band Internet of Things (NB-IoT), Long Range Radio (Rola), Zigbee, Micro Air Vehicle Link (MavLink), WIFI, Near Field Communication (NFC), General packet radio service (GPRS), Global System for Mobile Communications (GSM) and Ethernet.

Optionally, the processor 5, the communicator 9, and the LED touch screen 8 may be integrated together. In addition, all or part of the processor 5, the communicator 9, and the LED touch screen 8 may be integrated together or implemented independently. In the implementation process, each operation of the above method or each module may be completed by a hardware integrated logic circuit in the processor 5 or instructions in a form of software.

Optionally, the LED touch screen 8 is electrically connected with a power supply unit. And the power supply unit supplies electric power to the processor 5, the communicator 9, and the LED touch screen 8. The power supply unit includes dry batteries or rechargeable batteries.

In an embodiment, in order to increase the dispersion of tensile forces on the user's teeth and the user's comfort, materials of the upper bite pad 2 and the lower bite pad 3 include one or a combination of foam, polylactic acid, nylon plastic, photosensitive resin, silicone, rubber, latex, acrylonitrile butadiene styrene (ABS) plastic, polyvinyl chloride (PVC) plastics, silicone resins, and acrylic-based resins.

As described above, the present disclosure provides an intelligent mouth opening rehabilitation device. The intelligent mouth opening rehabilitation device includes: a body, an end of the body being provided with an upper bite pad and a lower bite pad; a pressure sensor, provided in the upper bite pad and/or the lower bite pad, for detecting pressure data on the upper bite pad and/or the lower bite pad; a stepping motor, for driving the upper bite pad and/or the lower bite pad to swing longitudinally; and a processor, for controlling the stepping motor to operate or stop according to the pressure data and/or a preset training instruction, thereby driving the upper bite pad and/or the lower bite pad to swing longitudinally or to stop.

Compared with traditional mouth opening rehabilitation devices that require manual measurement of the opening angle or opening distance per use and require the need to manually control the training duration, the intelligent mouth opening rehabilitation device in the present disclosure controls automatically the upper bite pad and/or the lower bite pad to swing longitudinally, so that the mouth opening training is more accurate, more scientific and more controllable. Therefore, the rehabilitation effect can be greatly improved by using the intelligent mouth opening rehabilitation device in the present disclosure.

As described above, the present disclosure effectively overcomes various defects in the prior art and has a high industrial value.

The foregoing embodiments only describe several implementations of the present disclosure, and their description is specific and detailed, but cannot therefore be understood as a limitation to the patent scope of the present disclosure. It should be noted that, a person of ordinary skill in the art may further make several variations and improvements without departing from the concept of the present disclosure, which shall fall within the protection scope of the present disclosure. Therefore, the protection scope of the present disclosure shall be subject to the appended claims.

1. An intelligent mouth opening rehabilitation device, comprising:
a body, wherein an end of the body is provided with an upper bite pad and a lower bite pad;
a pressure sensor, provided in the upper bite pad and/or the lower bite pad, for detecting pressure data on the upper bite pad and/or the lower bite pad;
a stepping motor, for driving the upper bite pad and/or the lower bite pad to swing longitudinally; and
a processor, for controlling the stepping motor to operate or stop according to the pressure data and/or a preset training instruction, thereby driving the upper bite pad and/or the lower bite pad to swing longitudinally or to stop;
wherein the processor controlling the stepping motor to operate or stop according to the pressure data comprises: the processor compares the pressure data with a preset pressure threshold in real time; if the pressure data are lower than the preset pressure threshold, the processor controls the stepping motor to operate forward, to enable the upper bite pad to swing up a distance and/or the lower bite pad to swing down a distance, then returns to the first operation; and if the pressure data are not lower than the preset pressure threshold, the processor controls the stepping motor to stop operating, to enable the upper bite pad and/or the lower bite pad to stop swinging; wherein the preset pressure threshold is set by testing a patient's pain performance in combination with a physician's observations when the physician instructs the patient on the use of the mouth opening rehabilitation device in an early stage;
wherein the processor controlling the stepping motor to operate or stop according to the pressure data further comprises: the pressure sensor detects the pressure data in real time to determine whether the pressure data reach a second pressure threshold; and if the pressure data reach the second pressure threshold, the processor controls the stepping motor to operate reversely, thereby enabling the upper bite pad to swing down a distance and/or the lower bite pad to swing up a distance, then returns to the first operation; wherein the second pressure threshold is greater than the preset pressure threshold, so that the patient increases a pressure exerted on the upper bite pad and the lower bite pad through upper and lower jaws of a mouth of the patient to reduce an opening angle between the upper bite pad and the lower bite pad, when the patient wants to interrupt a mouth opening training or when the patient wants to reduce the opening angle due to increasing soreness of the oral-jaw muscles.

2. (canceled)

3. (canceled)

4. The intelligent mouth opening rehabilitation device according to claim 1, wherein the preset training instruction comprises: a preset training duration, and a preset opening angle or a preset opening distance between the upper bite pad and the lower bite pad.

5. The intelligent mouth opening rehabilitation device according to claim 4, wherein an angle sensor is provided in the upper bite pad and/or the lower bite pad and used to collect and characterize the opening angle between the upper bite pad and the lower bite pad; or an infrared rangefinder is provided in the upper bite pad and/or the lower bite pad and used to collect and characterize an opening distance between the upper bite pad and the lower bite pad.

6. The intelligent mouth opening rehabilitation device according to claim 5, wherein the processor controlling the stepping motor to operate or stop according to the preset opening angle or the preset opening distance in the preset training instruction comprises:
the processor controls the stepping motor to operate forward according to the preset opening angle or the preset opening distance, to enable the upper bite pad to swing up a distance and/or the lower bite pad to swing down a distance; and
when the angle sensor detects that the opening angle between the upper bite pad and the lower bite pad reaches the preset opening angle, or when the infrared rangefinder detects that the opening distance between the upper bite pad and the lower bite pad reaches the preset opening distance, the processor controls the stepping motor to stop operating.

7. The intelligent mouth opening rehabilitation device according to claim 4, wherein the processor controlling the stepping motor to operate or stop according to the preset training duration in the preset training instruction, comprises:
when the stepping motor starts and enters a stop state, a countdown is carried out based on the preset training duration; and
when a timer detects that the training duration has been reached, a speaker emits a prompt tone, and the processor controls the stepping motor to operate reversely, so that the upper bite pad swings down to its starting position and/or the lower bite pad swings up to its starting position.

8. The intelligent mouth opening rehabilitation device according to claim 4, wherein the intelligent mouth opening rehabilitation device further comprises:
a light emitting diode touch screen, located on one side of the body, used to display real-time pressure data, a training duration, training times, and an opening angle or an opening distance, and/or, used to manually set preset training times and the preset opening angle or the preset opening distance; and
a communicator, communicating with an external device, used to send the real-time pressure data, the training duration, the training times, and the opening angle or the opening distance to the external device, and/or, used to receive the preset training times and the preset opening angle or the preset opening distance from the external device.

9. The intelligent mouth opening rehabilitation device according to claim 8, wherein a communication mode of the communicator comprises a wired communication mode and/or a wireless communication mode:
wherein the wired communication mode comprises one or a combination of USB1.0/2.0/3.x, Micro USB, Mini USB, serial interface and parallel interface;
wherein the wireless communication mode comprises one or a combination of 2G/3G/4G/5G, Bluetooth, Infrared, NB-IoT, Rola, Zigbee, MavLink, WIFI, NFC, GPRS, GSM and Ethernet.

10. The intelligent mouth opening rehabilitation device according to claim 1, wherein the upper bite pad and the lower bite pad are both provided with a corresponding stepping motor respectively; or, one of the upper bite pad and the lower bite pad is provided with a corresponding stepping motor and the other of the upper bite pad and the lower bite pad is fixed.
